(19) United States
(12) Patent Application Publication
(10) Pub. No.: US 2018/0317414 A1
(43) Pub. Date: Nov. 8, 2018

(54) NON-TRANSGENIC HAPLOID INDUCER LINES IN CUCURBITS

(71) Applicant: RIJK ZWAAN ZAADTEELT EN ZAADHANDEL B.V., De Lier (NL)

(72) Inventors: Cornelis Maria Petrus VAN DUN, De Lier (NL); Cecilia Lucia Clara LELIVELT, De Lier (NL); Sara MOVAHEDI, De Lier (NL)

(21) Appl. No.: 15/774,779

(22) PCT Filed: Nov. 8, 2016
(86) PCT No.: PCT/EP2016/076985

§ 371 Date: May 9, 2018

(30) Foreign Application Priority Data
Nov. 9, 2015 (NL) 2015752

(51) Int. Cl. A01H 1/08

(52) U.S. Cl. CPC A01H 1/08

(57) ABSTRACT

The present invention relates to a mutant plant of the Cucurbitaceae family comprising a modified CENP-C gene, which mutant plant when crossed to a wild-type plant having 2n chromosomes produces progeny, at least 0.1% of which have n chromosomes. The modification comprises for example a mutation in the CENP-C gene that leads to the occurrence of a premature stop codon or to a non-conservative amino acid change in the C-terminal region of the encoded protein. The plant is for example a Cucumis sativus plant and the modified CENP-C gene encodes a modified CENP-C protein that comprises at least one not-tolerated amino acid change or a premature stop codon in the C-terminal region, when compared to the CENP-C protein of SEQ ID No:1, in particular a mutation selected from E674K and S650N. The plant may also be a Cucumis melo plant wherein the modified CENP-C gene encodes a modified CENP-C protein that comprises at least one not-tolerated amino acid change or a premature stop codon in the C-terminal region, when compared to the CENP-C protein of SEQ ID No:2.

The present invention relates to a mutant plant of the Cucurbitaceae family that can be used as a non-transgenic haploid inducer line. The invention further relates to parts of the plants, such as the fruits, to seeds and to other propagation material, and to progeny of the plants.

In plant breeding, the main goal is to combine as many desirable traits as possible in a single genome, while at the same time eliminating as many undesirable traits as possible. This is a slow process that requires the crossing of many individual lines, evaluating the outcome of such crosses during the course of several growth seasons, and selecting promising offspring for further research. Often a selected line displays a few very good characteristics (such as, for example, larger fruits, drought tolerance, disease resistance, faster germination capacity, etc), but also many suboptimal properties that would not be accepted by the consumer and/or by the plant grower. The interesting characteristics of the selected line then need to be introduced into a commercially acceptable genetic background, without losing any of the commercially important traits, to eventually end up with a pure breeding line, in which all desired traits are genetically fixed. This endeavour typically requires multiple generations of backcrossing, because genetically unlinked traits tend to segregate away from each other, and this is therefore a very slow process. Depending on the average generation time (from seed to seed) of the species the creation of a new plant variety may take between 8 and 20 years. A pure breeding line can e.g. be used as a parent of a hybrid variety. Two inbred lines (whose genomes are highly homozygous) are crossed to each other, and the resulting hybrid seeds are sold. Hybrid lines usually display a combination of the superior characteristics of their parents, and they often outperform both their parents due to the high heterozygosity of their genome (hybrid vigour).

Plant breeding can be accelerated through the use of Doubled Haploid (DH) lines, which have a fully homozygous genome within a single generation. An important advantage of DHs is that they are fertile and can be sexually propagated indefinitely.

DHs can be created from the spores of a plant by means of e.g. androgenesis or gynogenesis protocols, or through the use of haploid inducer systems. The genome of these haploid plants is subsequently doubled, which explains why they are completely homozygous. Genome doubling can either occur spontaneously, or it can be induced through the addition of mitosis-blocking chemicals such as colchicine, oryzalin or trifluralin. This leads to the formation of doubled haploid plants (DH plants, DHs), which are able to produce seeds. In this manner the doubled haploid lines are immortalised. Each DH line represents one specific combination of traits derived from the parents of the starting plant, resulting from the reshuffling of all genetically unlinked traits during meiosis.

DHs can be produced from the spores of a starting plant by first creating haploid plants of the spores by means of androgenesis, such as microspore culture or anther culture, by gynogenesis, or by inducing the loss of maternal or paternal chromosomes from a zygote resulting from a fertilisation event, and then doubling the genome of the haploid plants thus obtained. The skilled person is very familiar with these methods of DH production, and he knows which method works best in his favourite plant species. Genome doubling may occur spontaneously, or it may be induced by the application of chemicals, such as colchicine, oryzalin or trifluralin. These chemicals disrupt spindle formation during mitosis, and are typically used for the blocking of mitosis.

The loss of maternal chromosomes from a zygote resulting from a fertilisation event can be induced by using a haploid inducer line as the female in a cross. Haploid inducer systems have been described in various plant species, for example when the female crossing partner is a plant of a different species than the male crossing partner. In interspecific crosses, loss of the genome of one of the parents has often been observed, such as in the cross between wheat and pearl millet, between barley and Hordeum bulbosum, and between tobacco (Nicotiana tabacum) and Nicotiana africana.

For members of the Cucurbitaceae family, protocols are available for the efficient in vitro production of DHs (see e.g. Gałązka & Niemirowicz-Szczytt 2013, Folia Hort. 25: 67-78; U.S. Pat. No. 5,492,827). However, DH protocols are not applicable to all genotypes, and several types of Cucurbits are not amenable to standard in vitro haploid induction techniques. It has not been possible to obtain DHs in vivo, as interspecific crosses leading to the loss of one of the parental genomes have not been described. Producing DHs in vivo has clear logistic advantages over the in vitro approaches: it is less labour-intensive, and it does not require a cell biology laboratory or controlled growth facilities for the sterile cultivation of plant material.

It is therefore an object of the current invention to provide an in vivo haploid inducer system for plants belonging to the Cucurbitaceae family.

In the literature, an in vivo system for obtaining haploid plants through genome elimination has been described for Arabidopsis thaliana. This system is based on the transgenic expression of a recombinantly altered CENH3 (centromeric histone H3) polypeptide in a plant having a corresponding inactivated endogenous CENH3 gene (Maruthachalam Ravi & Simon W. L. Chan; Haploid plants produced by centromere-mediated genome elimination; Nature 464 (2010), 615-619; US-2011/0083202; WO2011/044132). CENH3 is a centromeric histone protein that is part of the kinetochore complex, and it plays an important role in chromosome segregation during mitosis and meiosis. CENH3 consists of a highly variable N-terminal tail domain and a conserved histone fold domain (HFD). Swapping the N-terminal tail domain of Arabidopsis CENH3 with that of another histone and the concurrent fusion to Green Fluorescent Protein (GFP) results in a situation wherein Arabidopsis plants expressing this recombinant fusion protein are partially sterile. When crossed to a wild-type Arabidopsis plant, the chromosomes of the parent expressing this recombinant fusion protein missegregate during embryogenesis, resulting in the elimination of the corresponding parental genome and the production of haploid plants whose chromosomes were solely derived from the wild-type parent. Genome doubling can subsequently be achieved as described above. CENH3 appears to be an essential gene, as null mutants in Arabidopsis display embryonic lethality.

The DHs produced by this approach are however considered to be transgenic (receiving a Genetically Modified Organism—GMO—status), according to the current legislation in e.g. Europe, even though they themselves do not contain a transgenic construct. For any line with a GMO status to receive approval for commercial use and animal and/or human consumption, it needs to undergo very extensive regulatory procedures, which are tremendously expensive and time-consuming. Moreover, in important parts of the worldwide food market, transgenic food is not allowed for human consumption, and not appreciated by the public.

It is therefore a further object of the current invention to provide an in vivo haploid inducer system for plants belonging to the Cucurbitaceae family, that gives rise to non-transgenic plants that can be commercially sold without a need for regulatory approval.

In the research leading to the present invention, plants of the Cucurbitaceae family were developed with mutations in the CENP-C (centromere protein C) gene. CENP-C is known to bind centromeric DNA, similarly to CENH3. It is characterized by the presence of a highly conserved domain of 24 amino acids, known as the CENP-C motif, which is usually present in the C-terminus of the protein, but the rest of the CENP-C protein sequence is not well conserved across different species.

It was surprisingly found that these mutants when crossed to a wild-type plant having 2n chromosomes produce progeny, at least 0.1% of which have n chromosomes.

The present invention thus provides a mutant plant of the Cucurbitaceae family comprising a modified CENP-C gene, which mutant plant when crossed to a wild-type plant having 2n chromosomes produces progeny, at least 0.1% of which have n chromosomes. The mutant plant of the invention can either be used as a female parent or as a male parent in a cross, and in both cases haploid progeny can be obtained.

The invention further relates to parts of the plants, such as seeds and to other propagation material, and to progeny of the plants. The parts, seeds, propagation material and progeny comprise the said mutation in their genome.

Suitably, the modified CENP-C gene of the present invention is not naturally occurring, and it comprises a mutation that has been induced by man. Mutations may be introduced into a DNA sequence of a plant genome by a number of methods known in the prior art. Random mutagenesis comprises the use of chemical compounds to induce mutations (such as ethyl methanesulfonate, nitrosomethylurea, hydroxylamine, proflavine, N-methyl-N-nitrosoguanidine, N-ethyl-N-nitrosourea, N-methyl-N-nitro-nitrosoguanidine, diethyl sulfate, ethylene imine, sodium azide, formaline, urethane, phenol and ethylene oxide), the use of physical means to induce mutations (such as UV-irradiation, fast-neutron exposure, X-rays, gamma irradiation), and the insertion of genetic elements (such as transposons, T-DNA, retroviral elements). Mutations may also be introduced in a targeted, controlled manner, by means of homologous recombination, oligonucleotide-based mutation induction, zinc-finger nucleases (ZFNs), transcription activator-like effector nucleases (TALENs) or Clustered Regularly Interspaced Short Palindromic Repeat (CRISPR) systems (such as CRISPR-Cas9 or CRISPR-Cpf1).

The presence of a mutation in a plant genome may be detected by a number of different techniques known in the prior art, including but not limited to DNA-sequencing, RNA-sequencing, SNP microarray, Restriction Fragment Length Polymorphism (RFLP), Invader® assay, KASP™ assay, TaqMan™ assay.

The term “modified CENP-C gene” refers to a CENP-C gene that is a non-naturally occurring variant of a naturally-occurring (wild-type) CENP-C gene, which comprises at least one non-synonymous nucleotide change relative to a corresponding wild-type CENP-C gene and which encodes a modified CENP-C protein. A non-synonymous nucleotide change is a point mutation in a coding nucleotide sequence that alters the amino acid sequence of the protein for which it codes. This can be either a missense mutation, which is a point mutation in which a single nucleotide change results in a codon that codes for a different amino acid than in the corresponding wild-type sequence, or it can be a non-sense mutation, which is a point mutation in which a single nucleotide change results in the change of a codon to a premature stop codon. A missense mutation leads to the expression of a modified CENP-C protein with at least one amino acid change when compared to the corresponding wild-type protein, and a non-sense mutation leads to the expression of a modified CENP-C protein that is truncated when compared to the corresponding wild-type protein.

The term “modified CENP-C protein” refers to a CENP-C protein that is a non-naturally occurring variant of a naturally-occurring (wild-type) CENP-C protein, which comprises at least one amino acid change or a premature stop codon, when compared to the corresponding wild-type protein sequence.

The modified CENP-C gene of the invention suitably comprises at least one mutation compared to an otherwise identical naturally occurring CENP-C gene, which at least one mutation gives rise to at least one amino acid change in the encoded protein or to the occurrence of a premature stop codon in the encoded protein.

In one embodiment the modification comprises a mutation that leads to a modification in the C-terminal region of the CENP-C protein (which is shown in FIG. 2), which mutation impairs the function of the encoded CENP-C protein. For the purpose of this invention, the C-terminal region of the CENP-C protein is defined as the last 85 amino acids at the C-terminal end of the CENP-C protein sequence, as shown in FIG. 2. For example, in the CENP-C sequence from cucumber (SEQ ID No:1), the C-terminal region comprises amino acid positions 646 until 730. The C-terminal region comprises the 24 amino acid long CENP-C motif, which is characteristic for CENP-C proteins and which has been underlined and printed in bold in the alignment of FIG. 2.

The modified CENP-C gene in said mutant plant suitably comprises at least one mutation compared to an otherwise identical naturally occurring CENP-C gene, which at least one mutation gives rise to at least one amino acid change in the encoded protein or to the occurrence of a premature stop codon in the encoded modified CENP-C protein.

In a preferred embodiment, the modification in the modified CENP-C protein comprises a mutation in the C-terminal region (FIG. 2), which mutation affects the function of the encoded CENP-C protein. In one embodiment said mutation is a non-sense mutation, i.e. it causes the occurrence of a premature stop-codon (TAA, TAG or TGA), leading to the expression of a shorter, truncated version of the encoded protein. In another embodiment said mutation causes an amino acid change in the encoded protein, such that the normal function of the encoded protein is impaired.

Preferably, the modified CENP-C protein comprises an amino acid change that is predicted to be not tolerated in view of the biological function of the protein. The effect of an amino acid substitution in the context of a given protein can be predicted in silico, e.g. with SIFT (Ng and Henikoff, 2001, Genome Res. 11: 863-874).

A “not tolerated” amino acid change may occur when an amino acid is replaced by another amino acid that has different chemical properties, i.e. a non-conservative amino acid substitution, also termed a non-conservative amino acid change (for example, when a hydrophobic, non-polar amino acid such as Ala, Val, Leu, Ile, Pro, Phe, Trp or Met is replaced by a hydrophilic, polar amino acid, such as Gly, Ser, Thr, Cys, Tyr, Asn or Gln, or when an acidic, negatively charged amino acid such as Asp or Glu is replaced by a basic, positively charged amino acid, such as Lys, Arg or His).

In one embodiment said mutation in the C-terminal region of the CENP-C protein causes the occurrence of a premature stop codon (TAA, TAG or TGA) in the coding sequence, leading to the expression of a shorter, truncated version of the encoded protein. In another embodiment said mutation in the C-terminal region of the CENP-C protein causes an amino acid change in the encoded protein, such that the normal function of the encoded protein is impaired.

In one embodiment, the present invention provides a plant of the Cucurbitaceae family comprising a non-conservative amino acid change in the C-terminal region of the CENP-C protein. With reference to the sequence of CENP-C in cucumber (SEQ ID No:1), said non-conservative amino acid change may occur at position 646 (S), position 647 (R), position 648 (R), position 649 (Q), position 650 (S), position 651 (L), position 652 (A), position 653 (G), position 654 (A), position 655 (G), position 656 (T), position 657 (T), position 658 (W), position 659 (Q), position 660 (S), position 661 (G), position 662 (V), position 663 (R), position 664 (R), position 665 (S), position 666 (T), position 667 (R), position 668 (F), position 669 (K), position 670 (T), position 671 (R), position 672 (P), position 673 (L), position 674 (E), position 675 (Y), position 676 (W), position 677 (K), position 678 (G), position 679 (E), position 680 (R), position 681 (L), position 682 (L), position 683 (Y), position 684 (G), position 685 (R), position 686 (V), position 687 (H), position 688 (E), position 689 (S), position 690 (L), position 691 (T), position 692 (T), position 693 (V), position 694 (I), position 695 (G), position 696 (L), position 697 (K), position 698 (Y), position 699 (V), position 700 (S), position 701 (P), position 702 (A), position703 (K), position 704 (G), position 705 (N), position 706 (G), position 707 (K), position 708 (P), position 709 (T), position 710 (M), position 711 (K), position 712 (V), position 713 (K), position 714 (S), position 715 (L), position 716 (V), position 717 (S), position 718 (N), position 719 (E), position 720 (Y), position 721 (K), position 722 (D), position 723 (L), position 724 (V), position 725 (E), position 726 (L), position 727 (A), position 728 (A), position 729 (L), or position 730 (H), or at a corresponding amino acid position in the orthologous CENP-C protein of another Cucurbitaceae species.

In one embodiment, the invention relates to a mutant cucumber plant expressing a mutated CENP-C protein with an E (glutamic acid, Glu) to K (lysine, Lys) amino acid substitution at position 29 of the C-terminus (numbering according to the C-terminal region sequence from cucumber (Cterm_CENPC_cucumber) in FIG. 2). This mutation has been caused by a G to A transition in the coding sequence. Because this mutation occurred at position 674 of the cucumber CENP-C protein of SEQ ID No:1, it was termed E674K.

In another embodiment, the invention relates to a mutant cucumber plant expressing a mutated CENP-C protein with a S (serine, Ser) to N (asparagine, Asn) amino acid substitution at position 5 of the C-terminus (numbering according to the C-terminal region sequence from cucumber (Cterm_CENPC_cucumber) in FIG. 2), due to a G to A transition in the coding sequence. Because this mutation occurred at position 650 of the cucumber CENP-C protein of SEQ ID No:1, it was termed S650N.

In another embodiment, the present invention provides a plant of the Cucurbitaceae family comprising a premature stop codon in the C-terminal region of the CENP-C protein, for which the sequences from cucumber and melon are presented in FIG. 2. With reference to the sequence of CENP-C in cucumber (SEQ ID No:1), mutagenesis with EMS or another alkylating chemical mutagen, which typically causes G to A and C to T transitions, may induce premature stop codons in the C-terminal region at position 649 (Q, encoded by CAA, which may mutate to TAA), at position 658 (W, encoded by TGG, which may mutate to TGA), at position 659 (Q, encoded by CAA, which may mutate to TAA), at position 671 (R, encoded by CGA, which may mutate to TGA), and at position 676 (W, encoded by TGG, which may mutate to TGA).

The present invention thus provides a mutant plant of the Cucurbitaceae family comprising a modified CENP-C gene, which mutant plant when crossed to a wild-type plant having 2n chromosomes produces progeny, at least 0.1% of which have n chromosomes, wherein said mutation leads to the occurrence of a premature stop codon or to a non-conservative amino acid change, preferably in the C-terminal region of the CENP-C gene.

The present invention further provides a mutant cucumber plant comprising a modified CENP-C gene that encodes a modified CENP-C protein that comprises at least one non-conservative amino acid change or a premature stop codon, preferably in the C-terminal region, when compared to the CENP-C protein of SEQ ID No:1, which mutant cucumber plant when crossed to a wild-type cucumber plant having 2n chromosomes produces progeny, at least 0.1% of which have n chromosomes.

The invention also provides a mutant melon plant comprising a modified CENP-C gene that encodes a modified CENP-C protein that comprises at least one non-conservative amino acid change or a premature stop codon, preferably in the C-terminal region, when compared to the CENP-C protein of SEQ ID No:2, which mutant melon plant when crossed to a wild-type melon plant having 2n chromosomes produces progeny, at least 0.1% of which have n chromosomes.

The wild-type coding DNA-sequences (CDS) of CENP-C from cucumber and melon can be found under SEQ ID No:3 and 4, respectively.

The present invention also relates to the use of said mutant plants for the production of haploid or doubled haploid plants.

The present invention further relates to a method for the production of haploid or doubled haploid plants, comprising:

a) providing a mutant plant of the Cucurbitaceae family according to the present invention;

b) crossing said mutant plant as one parent with a wild-type plant of the same species as the other parent;

c) growing progeny seeds from the cross;

d) selecting progeny plants with a haploid genome that only comprises chromosomes from the wild-type parent, and progeny plants with a diploid genome that only comprises chromosomes from the wild-type parent;

e) optionally doubling the genome of haploid progeny plants selected in step d).

The present invention also relates to haploid and doubled haploid plants of the Cucurbitaceae family, obtainable by the above-described method.

The present invention also provides a plant belonging to the Cucurbitaceae family harbouring at least one mutation in another centromeric histone protein-encoding gene, in addition to the at least one mutation in the CENP-C gene.

In one embodiment, the at least one mutation in another centromeric histone protein-encoding gene is in the CENH3 (centromeric histone H3) gene. The present invention thus also provides a mutant plant of the Cucurbitaceae family, comprising a modified CENP-C gene and a modified CENH3 gene, which mutant plant when crossed to a wild-type plant having 2n chromosomes produces progeny, at least 0.1% of which have n chromosomes.

Suitably, the modified CENH3 gene in said mutant plant comprises at least one mutation compared to an otherwise identical naturally occurring CENH3 gene, which at least one mutation gives rise to at least one non-conservative amino acid change in the Histone Fold Domain of the encoded modified CENH3 protein or to the occurrence of a premature stop codon in the encoded modified CENH3 protein. Suitably, the modified CENP-C gene in said mutant plant comprises at least one mutation compared to an otherwise identical naturally occurring CENP-C gene, wherein said mutation leads to the occurrence of a premature stop codon or to a non-conservative amino acid change, preferably in the C-terminal region of the encoded modified CENP-C protein.

The present invention further provides a mutant cucumber plant comprising a modified CENH3 gene that encodes a modified CENH3 protein that comprises at least one non-conservative amino acid change or a premature stop codon, preferably in the Histone Fold Domain, when compared to the CENH3 protein of SEQ ID No:5, and a modified CENP-C gene that encodes a protein that comprises at least one non-conservative amino acid change or a premature stop codon, preferably in the C-terminal region, when compared to the CENP-C protein of SEQ ID No:1, which mutant cucumber plant when crossed to a wild-type cucumber plant having 2n chromosomes produces progeny, at least 0.1% of which have n chromosomes. The C-terminal region of CENP-C starts at position 646 in the sequence of SEQ ID No:1, and it has been underlined in that sequence. The Histone Fold Domain of CENH3 has been underlined in SEQ ID No:5. Preferably, the modified CENH3 and CENP-C proteins each comprise at least one amino acid change that is predicted to be not tolerated in view of the biological function of the respective protein, as predicted with SIFT analysis (Ng and Henikoff, 2001, Genome Res. 11: 863-874).

The present invention also provides a mutant melon plant comprising a modified CENH3 gene that encodes a modified CENH3 protein that comprises at least one non-conservative amino acid change or a premature stop codon, preferably in the Histone Fold Domain, when compared to the CENH3 protein of SEQ ID No:6, and a modified CENP-C gene that encodes a protein that comprises at least one non-conservative amino acid change or a premature stop codon, preferably in the C-terminal region, when compared to the CENP-C protein of SEQ ID No:2, which mutant cucumber plant when crossed to a wild-type cucumber plant having 2n chromosomes produces progeny, at least 0.1% of which have n chromosomes. The C-terminal region starts at position 645 in the sequence of SEQ ID No:2, and it has been underlined in that sequence. The Histone Fold Domain of CENH3 has been underlined in SEQ ID No:6. Preferably, the modified CENH3 and CENP-C proteins each comprise at least one amino acid change that is predicted to be not tolerated in view of the biological function of the respective protein, as predicted with SIFT analysis (Ng and Henikoff, 2001, Genome Res. 11: 863-874).

The present invention can be applied in plants belonging to the Cucurbitaceae family. This plant family comprises various commercially important genera, such as Cucurbita, Cucumis, Lagenaria, Citrullus, Luffa, Benincasa, Momordica, and Trichosantes. These genera comprise, among others, the following vegetable species: Cucumis spp (cucumber, melon, gherkin), Cucurbita spp (zucchini, pumpkin, squash), Citrullus spp (watermelon), Benincasa cerifera (wax gourd), Lagenaria leucantha (bottle gourd), Luffa acutangula (ridge gourd), Luffa cylindrica (sponge gourd), Momordica charantia (bitter gourd), and Trichosantes cucumerina (snake gourd).

The invention will be further illustrated in the following Examples. In these Examples reference is made to the following figures.

### FIGURES

FIG. 1: alignment of CENP-C protein sequences from melon (Cucumis melo) and cucumber (Cucumis sativus). Stars below the alignment indicate amino acid positions that are identical in the proteins from all four species. Sequence conservation is very high in the C-terminal region of the CENP-C protein, which contains the CENP-C motif (see also FIG. 2).

FIG. 2: alignment of the C-terminal region of CENP-C protein sequences from melon (Cucumis melo) and cucumber (Cucumis sativus). The CENP-C motif is underlined and printed in bold.

### EXAMPLES

### Example 1

### Identification of CENP-C Orthologues in Cucurbitaceae

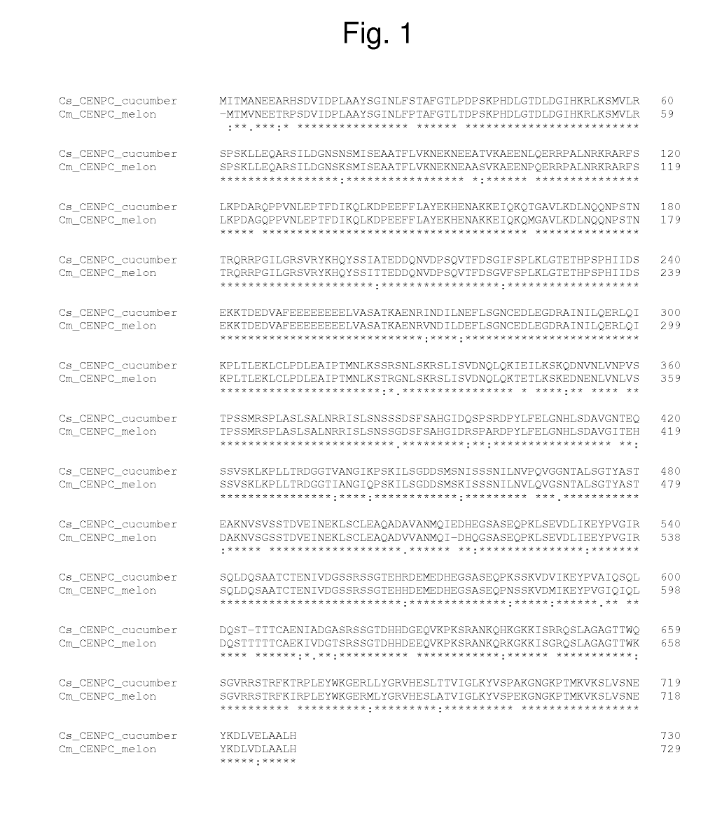

Orthologues of the CENP-C gene were identified in Cucurbitaceae species by using a Blasting programme (TBLASTN) to compare the highly conserved CENP-C motif sequence with the sequences of crop species of the Cucurbitaceae family. This search resulted in the identification of CENP-C genes in cucumber and melon. FIG. 1 shows the alignment of these two sequences.

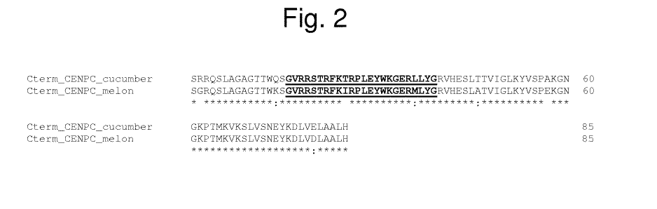

Comparison of the sequences revealed that the C-terminal region of CENP-C is very well conserved in these two commercially important vegetable species belonging to the Cucurbitaceae family. Only for seven of the 85 positions in the C-terminal region a difference was observed. This is shown in the alignment of FIG. 2. This high degree of conservation indicates that any mutation that is found to cause a haploid-inducer phenotype in one of these species can reliably be expected to cause the same phenotype in the other species. The information obtained from the study of a plant with mutated C-terminus in CENP-C of one of the Cucurbitaceae species can thus be directly translated to other Cucurbitaceae species.

### Example 2

Identification of Cenp-C Mutant Cucumber Plants with Haploid Inducer Phenotype

Plants of cucumber (Cucumis sativus) line KK 5735 were mutagenised with EMS (ethyl methanesulfonate). In a TILLING approach (Targeting Induced Local Lesions in Genomes), 6144 plants of the EMS-mutagenised population were subsequently screened for point mutations in the CENP-C gene. This screen resulted in the identification of a number of plants with mutations in the C-terminal region of CENP-C.

A cucumber plant expressing a mutated CENP-C protein with an E (glutamic acid, Glu) to K (lysine, Lys) amino acid substitution at position 29 of the C-terminus (numbering according to the C-terminal region sequence from cucumber (Cterm_CENPC_cucumber) in FIG. 2) was identified in this screen, which had been caused by a G to A transition in the coding sequence. Because this mutation occurred at position 674 of the cucumber CENP-C protein of SEQ ID No:1, it was termed E674K. This mutant plant was found to possess said mutation in a heterozygous state. After selfing, mutant plants were obtained that harboured the E674K mutation in a homozygous state, and these were used for further experimentation. The E674K mutation was predicted to be functionally not tolerated by SIFT analysis.

The homozygous E674K mutant plant was pollinated with pollen from a wild-type cucumber plant, which was genetically distinct from line KK 5735, such that a set of polymorphic molecular markers could be selected with which the two parents of the cross as well as their hybrid progeny could be unambiguously identified by means of molecular marker analysis of their genome. The fruits resulting from the cross were harvested, and seeds were collected and sown on agar medium (0.5×MS salts with 10 g L⁻¹ sucrose), and incubated at 25° C. in long-day conditions (16 hours light, 8 hours darkness).

When seedlings were big enough, tissue samples were taken from the cotyledons for molecular marker analysis. This analysis revealed that most of the progeny plants were hybrids of mother line KK 5735 and the genetically distinct father line, but about 1.4% of the progeny plants were shown to be genetically identical to the father line. These plants were transplanted to soil in the greenhouse for further analysis. Flow cytometry showed that most of these plantlets were haploid, although some of them had spontaneously doubled their genome and had become doubled haploids. The haploid progeny was treated with colchicine to induce genome doubling.

Another cucumber mutant identified in the screen comprised an S (serine, Ser) to N (asparagine, Asn) amino acid substitution at position 5 of the C-terminus (numbering according to the C-terminal region sequence from cucumber (Cterm_CENPC_cucumber) in FIG. 2), due to a G to A transition in the coding sequence. Because this mutation occurred at position 650 of the cucumber CENP-C protein of SEQ ID No:1, it was termed S650N. This mutant plant was found to possess said mutation in a heterozygous state. After selfing, mutant plants were obtained that harboured the S650N mutation in a homozygous state, and these were used for further experimentation. The S650N mutation was predicted to be functionally not tolerated by SIFT analysis.

The homozygous S650N mutant plant was pollinated with pollen from a wild-type cucumber plant, which was genetically distinct from line KK 5735, such that a set of polymorphic molecular markers could be selected with which the two parents of the cross as well as their hybrid progeny could be unambiguously identified by means of molecular marker analysis of their genome. The fruits resulting from the cross were harvested, and seeds were collected and sown on agar medium (0.5×MS salts with 10 g L⁻¹ sucrose), and incubated at 25° C. in long-day conditions (16 hours light, 8 hours darkness).

When seedlings were big enough, tissue samples were taken from the cotyledons for molecular marker analysis. This analysis revealed that most of the progeny plants were hybrids of mother line KK 5735 and the genetically distinct father line, but about 0.8% of the progeny plants were shown to be genetically identical to the father line. These plants were transplanted to soil in the greenhouse for further analysis. Flow cytometry showed that most of these plantlets were haploid, although some of them had spontaneously doubled their genome and had become doubled haploids. The haploid progeny was treated with colchicine to induce genome doubling.

SEQUENCES

SEQ ID No: 1
>CENPC_cucumber
MITMANEEARHSDVIDPLAAYSGINLFSTAFGTLPDPSKPHDLGTDLDGI
HKRLKSMVLRSPSKLLEQARSILDGNSNSMISEAATFLVKNEKNEEATVK
AEENLQERRPALNRKRARFSLKPDARQPPVNLEPTFDIKQLKDPEEFFLA
YEKHENAKKEIQKQTGAVLKDLNQQNPSTNTRQRRPGILGRSVRYKHQYS
SIATEDDQNVDPSQVTFDSGIFSPLKLGTETHPSPHIIDSEKKTDEDVAF
EEEEEEEELVASATKAENRINDILNEFLSGNCEDLEGDRAINILQERLQI
KPLTLEKLCLPDLEAIPTMNLKSSRSNLSKRSLISVDNQLQKIEILKSKQ
DNVNLVNPVSTPSSMRSPLASLSALNRRISLSNSSSDSFSAHGIDQSPSR
DPYLFELGNHLSDAVGNTEQSSVSKLKPLLTRDGGTVANGIKPSKILSGD
DSMSNISSSNILNVPQVGGNTALSGTYASTEAKNVSVSSTDVEINEKLSC
LEAQADAVANMQIEDHEGSASEQPKLSEVDLIKEYPVGIRSQLDQSAATC
TENIVDGSSRSSGTEHRDEMEDHEGSASEQPKSSKVDVIKEYPVAIQSQL
DQSTTTTCAENIADGASRSSGTDHHDGEQVKPKSRANKQHKGKKISRRQS
LAGAGTTWQSGVRRSTRFKTRPLEYWKGERLLYGRVHESLTTVIGLKYVS
PAKGNGKPTMKVKSLVSNEYKDLVELAALH
SEQ ID No: 2
>CENPC_melon
MTMVNEETRPSDVIDPLAAYSGINLFPTAFGTLTDPSKPHDLGTDLDGIH
KRLKSMVLRSPSKLLEQARSILDGNSKSMISEAATFLVKNEKNEAASVKA
EENPQERRPALNRKRARFSLKPDAGQPPVNLEPTFDIKQLKDPEEFFLAY
EKHENAKKEIQKQMGAVLKDLNQQNPSTNTRQRRPGILGRSVRYKHQYSS
ITTEDDQNVDPSQVTFDSGVFSPLKLGTETHPSPHIIDSEKKTDEDVAFE
EEEEEEELVASATKAENRVNDILDEFLSGNCEDLEGDRAINILQERLQIK
PLTLEKLCLPDLEAIPTMNLKSTRGNLSKRSLISVDNQLQKTETLKSKED
NENLVNLVSTPSSMRSPLASLSALNRRISLSNSSGDSFSAHGIDRSPARD
PYLFELGNHLSDAVGITEHSSVSKLKPLLTRDGGTIANGIQPSKILSGDD
SMSKISSSNILNVLQVGSNTALSGTYASTDAKNVSGSSTDVEINEKLSCL
EAQADVVANMQIDHQGSASEQPKLSEVDLIEEYPVGIRSQLDQSAATCTE
NIVDGSSRSSGTEHHDEMEDHEGSASEQPNSSKVDMIKEYPVGIQIQLDQ
STTTTTCAEKIVDGTSRSSGTDHHDEEQVKPKSRANKQRKGKKISGRQSL
AGAGTTWKSGVRRSTRFKIRPLEYWKGERMLYGRVHESLATVIGLKYVSP
EKGNGKPTMKVKSLVSNEYKDLVDLAALH
SEQ ID No: 3
>CENPC_cucumber_CDS
ATGATAACAATGGCGAACGAAGAAGCTCGACACTCCGATGTTATCGATCC
TCTTGCTGCTTATTCTGGTATCAATCTTTTTTCGACCGCATTTGGTACTT
TGCCGGATCCGTCAAAGCCACATGATCTTGGAACAGACCTCGACGGCATC
CACAAGCGCCTCAAATCCATGGTGTTAAGGAGTCCCAGTAAACTATTAGA
ACAGGCCAGATCAATTTTAGATGGCAACTCAAATTCGATGATATCTGAAG
CTGCCACATTTCTTGTGAAGAATGAGAAAAATGAGGAAGCTACAGTGAAG
GCAGAGGAAAATCTTCAAGAAAGAAGGCCGGCCTTAAACCGAAAGCGGGC
TAGGTTTTCTTTAAAACCCGATGCTAGGCAACCTCCTGTGAACTTGGAAC
CAACATTTGACATCAAACAATTGAAAGACCCCGAGGAGTTCTTTTTGGCC
TATGAAAAGCATGAAAATGCCAAAAAAGAAATCCAGAAGCAGACGGGAGC
AGTTTTAAAGGACTTGAACCAACAAAATCCGTCGACGAATACACGCCAGC
GTAGACCGGGGATTCTTGGAAGATCTGTTAGATACAAGCATCAATATTCA
TCAATAGCAACTGAAGATGATCAGAATGTAGATCCTTCTCAAGTGACATT
TGATTCAGGCATTTTCAGTCCATTGAAATTGGGCACAGAAACACACCCAA
GTCCACATATAATTGACTCAGAAAAGAAAACTGATGAAGATGTAGCCTTT
GAGGAGGAGGAGGAGGAGGAGGAGCTCGTTGCTTCAGCTACGAAGGCAGA
GAACAGAATAAATGATATTTTGAATGAATTTCTCTCTGGTAATTGTGAAG
ATCTAGAAGGTGATCGAGCCATCAACATATTACAGGAGCGCTTGCAGATT
AAACCTCTTACTTTAGAGAAATTATGCCTTCCAGATTTAGAAGCCATTCC
AACAATGAATTTGAAATCTTCAAGAAGCAATCTATCAAAGCGTAGTTTGA
TCAGTGTGGACAATCAGTTACAAAAGATAGAAATTTTGAAATCTAAGCAG
GACAATGTAAATTTGGTTAATCCTGTTTCTACACCATCATCAATGAGAAG
TCCATTGGCATCGTTATCAGCACTAAATAGACGGATTTCACTTTCAAATT
CATCAAGTGATTCATTTTCAGCTCATGGCATTGACCAATCTCCATCAAGA
GATCCTTACCTTTTTGAACTCGGTAATCACTTATCTGATGCAGTTGGTAA
TACAGAGCAGTCAAGCGTTTCTAAGTTGAAGCCACTTTTAACCAGAGATG
GTGGGACTGTAGCAAATGGAATTAAACCATCCAAAATTCTTTCTGGAGAT
GATTCCATGTCTAATATATCTTCAAGTAATATTTTAAATGTACCCCAAGT
TGGGGGCAATACTGCTTTAAGTGGAACTTATGCCAGCACGGAGGCTAAAA
ATGTTAGTGTCAGCAGCACAGACGTGGAAATAAATGAGAAATTGAGTTGT
CTTGAAGCCCAAGCAGATGCGGTGGCTAATATGCAGATTGAAGATCACGA
AGGATCAGCTTCTGAGCAACCAAAATTATCTGAGGTGGATCTAATCAAAG
AGTACCCGGTTGGCATTCGGAGTCAGTTGGATCAATCAGCTGCTACTTGT
ACTGAAAATATTGTTGATGGGTCATCTAGAAGCAGTGGTACAGAACACCG
CGATGAGATGGAAGATCATGAAGGATCAGCTTCTGAGCAACCAAAGTCAT
CTAAGGTGGATGTGATTAAAGAGTACCCAGTAGCCATTCAGAGTCAGTTG
GATCAATCAACTACTACTACTTGTGCTGAAAATATTGCCGATGGGGCATC
TAGAAGCAGTGGAACGGATCACCATGATGGGGAACAGGTCAAGCCAAAAT
CTCGTGCAAACAAACAACACAAAGGCAAAAAGATTTCTCGGAGGCAAAGC
CTTGCAGGTGCTGGTACAACGTGGCAAAGTGGGGTGAGAAGAAGTACCAG
GTTCAAAACACGACCCTTGGAGTACTGGAAAGGTGAAAGGCTGTTGTACG
GACGTGTACATGAGAGCCTGACGACAGTAATTGGGTTGAAGTATGTGTCT
CCAGCAAAAGGAAATGGCAAACCAACCATGAAGGTGAAGTCTCTAGTCTC
CAATGAGTACAAAGATCTCGTCGAGTTAGCAGCCCTTCACTGA
SEQ ID No: 4
>CENPC_melon_CDS
ATGACAATGGTGAACGAAGAAACTCGACCCTCCGATGTAATCGATCCTCT
TGCTGCTTATTCTGGTATCAATCTCTTTCCGACCGCATTTGGTACTTTGA
CGGATCCGTCAAAGCCACATGATCTTGGAACAGACCTCGACGGCATCCAC
AAGCGCCTCAAATCCATGGTGTTAAGGAGTCCCAGTAAACTATTAGAGCA
GGCCAGATCAATATTAGATGGCAACTCAAAATCGATGATATCTGAAGCTG
CTACATTTCTCGTGAAGAATGAGAAAAATGAGGCAGCTTCTGTGAAGGCA
GAGGAAAATCCTCAAGAAAGAAGGCCGGCCTTAAACCGAAAGCGGGCTAG
GTTTTCTTTAAAACCTGATGCTGGGCAACCTCCTGTGAACTTGGAACCAA
CATTTGACATCAAACAATTGAAAGACCCTGAGGAGTTCTTTTTGGCCTAT
GAAAAGCATGAAAATGCCAAAAAAGAAATCCAAAAACAGATGGGAGCAGT
TTTAAAGGACTTGAACCAACAAAATCCATCGACAAATACACGCCAGCGTA
GACCAGGGATTCTTGGGAGATCTGTTAGATACAAGCATCAATATTCATCA
ATAACAACTGAAGATGATCAGAATGTAGATCCTTCTCAAGTGACATTTGA
TTCAGGTGTTTTCAGTCCATTGAAATTGGGCACAGAAACACACCCAAGTC
CACATATAATTGACTCAGAAAAGAAAACTGATGAAGATGTAGCCTTTGAG
GAGGAGGAGGAGGAGGAGGAGCTCGTTGCTTCAGCTACGAAGGCAGAGAA
CAGAGTAAATGATATTTTGGATGAATTTCTCTCTGGCAATTGTGAAGATC
TAGAAGGTGATCGAGCTATCAACATATTACAGGAGCGCTTGCAGATTAAA
CCCCTTACTTTAGAGAAATTATGCCTTCCAGATTTAGAAGCCATTCCAAC
AATGAATTTGAAATCTACAAGAGGCAATCTGTCAAAGCGTAGTTTGATCA
GTGTGGACAATCAGTTACAAAAGACAGAAACCTTGAAATCTAAGGAGGAC
AATGAAAATTTGGTTAATCTTGTTTCTACACCATCATCAATGAGAAGTCC
ATTGGCATCATTATCAGCCCTAAATAGACGAATTTCACTTTCAAATTCAT
CAGGTGATTCATTTTCAGCTCATGGCATCGACCGATCTCCAGCAAGAGAT
CCTTACCTTTTTGAACTCGGTAATCACTTATCTGATGCAGTTGGTATTAC
AGAGCATTCAAGCGTTTCTAAGTTGAAGCCACTTTTAACCAGAGATGGTG
GGACTATAGCAAATGGAATTCAACCATCCAAAATTCTTTCTGGAGACGAT
TCCATGTCTAAAATATCTTCAAGTAATATTTTAAATGTACTCCAAGTTGG
TAGCAATACTGCTTTAAGTGGAACTTATGCCAGCACAGATGCTAAAAATG
TTAGTGGGAGCAGCACAGACGTGGAAATAAATGAGAAATTAAGTTGTCTT
GAAGCCCAAGCAGATGTGGTGGCTAATATGCAGATAGATCACCAAGGATC
AGCTTCTGAGCAACCAAAATTATCTGAGGTGGATCTTATTGAAGAGTACC
CGGTTGGCATTCGGAGTCAGTTGGATCAATCAGCTGCTACTTGTACTGAA
AATATTGTTGATGGGTCGTCTAGAAGCAGTGGAACAGAACACCACGATGA
GATGGAAGATCACGAAGGATCAGCTTCTGAGCAACCAAACTCATCTAAGG
TGGATATGATTAAAGAGTACCCAGTCGGCATTCAGATTCAGTTGGATCAA
TCAACTACTACTACTACTTGTGCTGAAAAAATTGTCGATGGGACATCTAG
AAGCAGTGGAACGGATCACCATGATGAGGAACAGGTCAAGCCAAAATCTC
GTGCAAACAAACAACGTAAAGGCAAAAAGATTTCTGGGAGGCAAAGCCTT
GCAGGTGCTGGTACAACGTGGAAAAGTGGGGTGAGAAGAAGTACCAGGTT
CAAAATACGACCCTTGGAGTACTGGAAAGGTGAAAGGATGTTGTACGGAC
GTGTACATGAGAGCCTAGCGACAGTAATCGGGTTGAAGTATGTGTCTCCA
GAAAAAGGAAATGGCAAACCAACCATGAAGGTGAAATCTCTAGTCTCCAA
TGAGTACAAAGATCTCGTCGACTTAGCAGCCCTTCACTGA
SEQ ID No:5
>CENH3_cucumber
MARARHPPRRKSNRTPSGSGAAQSSPTAPSTPLNGRTQNVRQAQNSSSRT
IKKKKRFRPGTVALKEIRNLQKSWNLLIPASCFIRAVKEVSNQLAPQITR
WQAEALVALQEAAEDFLVHLFEDTMLCAIHAKRVTIMKKDFELARRLGGK
GRPW
SEQ ID No: 6
>CENH3_melon
MARARHPVQRKSNRTSSGSGAALSPPAVPSTPLNGRTQNVRKAQSPPSRT
KKKKIRFRPGTVALREIRNLQKSWNLLIPASCFIRAVKEVSNQLAPQITR
WQAEALVALQEAAEDFLVHLFEDTMLCAIHAKRVTIMKKDFELARRLGGK
GRPW

1. Mutant plant of the Cucurbitaceae family comprising a modified CENP-C gene, which mutant plant when crossed to a wild-type plant having 2n chromosomes produces progeny, at least 0.1% of which have n chromosomes.

2. Mutant plant as claimed in claim 1, wherein the modification comprises a mutation in the CENP-C gene that leads to the occurrence of a premature stop codon or to a non-conservative amino acid change in the C-terminal region of the encoded protein.

3. Mutant plant as claimed in claim 1, wherein the plant is a Cucumis sativus plant and the modified CENP-C gene encodes a modified CENP-C protein that comprises at least one not-tolerated amino acid change or a premature stop codon in the C-terminal region, when compared to the CENP-C protein of SEQ ID No:1, in particular a mutation selected from E674K and S650N.

4. Mutant plant as claimed in claim 1, wherein the plant is a Cucumis melo plant and the modified CENP-C gene encodes a modified CENP-C protein that comprises at least one not-tolerated amino acid change or a premature stop codon in the C-terminal region, when compared to the CENP-C protein of SEQ ID No:2.

5. Part of the mutant plant of claim 1, in particular the seeds and other propagation material, which part comprises the mutation in its genome.

6. Use of the mutant plant of claim 1 for the production of haploid or doubled haploid plants.

7. Method for the production of haploid or doubled haploid plants, comprising:
a) providing a mutant plant according to claim 1;
b) crossing said mutant plant as one parent with a wild-type plant of the same species as the other parent;
c) growing progeny seeds from the cross;
d) selecting progeny plants with a haploid genome that only comprises chromosomes from the wild-type parent, and progeny plants with a diploid genome that only comprises chromosomes from the wild-type parent;
e) optionally doubling the genome of haploid progeny plants selected in step d).

8. Doubled haploid plants obtainable by the method of claim 7.


SEQUENCE LISTING (8 sequences)

SEQ ID NO: 1 - protein, 730 aa, Cucumis sativus
MITMANEEARHSDVIDPLAAYSGINLFSTAFGTLPDPSKPHDLGTDLDGIHKRLKSMVLRSPSKLLEQARSILDGNSNSMISEAATFLVKNEKNEEATVKAEENLQERRPALNRKRARFSLKPDARQPPVNLEPTFDIKQLKDPEEFFLAYEKHENAKKEIQKQTGAVLKDLNQQNPSTNTRQRRPGILGRSVRYKHQYSSIATEDDQNVDPSQVTFDSGIFSPLKLGTETHPSPHIIDSEKKTDEDVAFEEEEEEEELVASATKAENRINDILNEFLSGNCEDLEGDRAINILQERLQIKPLTLEKLCLPDLEAIPTMNLKSSRSNLSKRSLISVDNQLQKIEILKSKQDNVNLVNPVSTPSSMRSPLASLSALNRRISLSNSSSDSFSAHGIDQSPSRDPYLFELGNHLSDAVGNTEQSSVSKLKPLLTRDGGTVANGIKPSKILSGDDSMSNISSSNILNVPQVGGNTALSGTYASTEAKNVSVSSTDVEINEKLSCLEAQADAVANMQIEDHEGSASEQPKLSEVDLIKEYPVGIRSQLDQSAATCTENIVDGSSRSSGTEHRDEMEDHEGSASEQPKSSKVDVIKEYPVAIQSQLDQSTTTTCAENIADGASRSSGTDHHDGEQVKPKSRANKQHKGKKISRRQSLAGAGTTWQSGVRRSTRFKTRPLEYWKGERLLYGRVHESLTTVIGLKYVSPAKGNGKPTMKVKSLVSNEYKDLVELAALH

SEQ ID NO: 2 - protein, 729 aa, Cucumis melo
MTMVNEETRPSDVIDPLAAYSGINLFPTAFGTLTDPSKPHDLGTDLDGIHKRLKSMVLRSPSKLLEQARSILDGNSKSMISEAATFLVKNEKNEAASVKAEENPQERRPALNRKRARFSLKPDAGQPPVNLEPTFDIKQLKDPEEFFLAYEKHENAKKEIQKQMGAVLKDLNQQNPSTNTRQRRPGILGRSVRYKHQYSSITTEDDQNVDPSQVTFDSGVFSPLKLGTETHPSPHIIDSEKKTDEDVAFEEEEEEEELVASATKAENRVNDILDEFLSGNCEDLEGDRAINILQERLQIKPLTLEKLCLPDLEAIPTMNLKSTRGNLSKRSLISVDNQLQKTETLKSKEDNENLVNLVSTPSSMRSPLASLSALNRRISLSNSSGDSFSAHGIDRSPARDPYLFELGNHLSDAVGITEHSSVSKLKPLLTRDGGTIANGIQPSKILSGDDSMSKISSSNILNVLQVGSNTALSGTYASTDAKNVSGSSTDVEINEKLSCLEAQADVVANMQIDHQGSASEQPKLSEVDLIEEYPVGIRSQLDQSAATCTENIVDGSSRSSGTEHHDEMEDHEGSASEQPNSSKVDMIKEYPVGIQIQLDQSTTTTTCAEKIVDGTSRSSGTDHHDEEQVKPKSRANKQRKGKKISGRQSLAGAGTTWKSGVRRSTRFKIRPLEYWKGERMLYGRVHESLATVIGLKYVSPEKGNGKPTMKVKSLVSNEYKDLVDLAALH

SEQ ID NO: 3 - protein, 2193 aa, Cucumis sativus
ATGATAACAATGGCGAACGAAGAAGCTCGACACTCCGATGTTATCGATCCTCTTGCTGCTTATTCTGGTATCAATCTTTTTTCGACCGCATTTGGTACTTTGCCGGATCCGTCAAAGCCACATGATCTTGGAACAGACCTCGACGGCATCCACAAGCGCCTCAAATCCATGGTGTTAAGGAGTCCCAGTAAACTATTAGAACAGGCCAGATCAATTTTAGATGGCAACTCAAATTCGATGATATCTGAAGCTGCCACATTTCTTGTGAAGAATGAGAAAAATGAGGAAGCTACAGTGAAGGCAGAGGAAAATCTTCAAGAAAGAAGGCCGGCCTTAAACCGAAAGCGGGCTAGGTTTTCTTTAAAACCCGATGCTAGGCAACCTCCTGTGAACTTGGAACCAACATTTGACATCAAACAATTGAAAGACCCCGAGGAGTTCTTTTTGGCCTATGAAAAGCATGAAAATGCCAAAAAAGAAATCCAGAAGCAGACGGGAGCAGTTTTAAAGGACTTGAACCAACAAAATCCGTCGACGAATACACGCCAGCGTAGACCGGGGATTCTTGGAAGATCTGTTAGATACAAGCATCAATATTCATCAATAGCAACTGAAGATGATCAGAATGTAGATCCTTCTCAAGTGACATTTGATTCAGGCATTTTCAGTCCATTGAAATTGGGCACAGAAACACACCCAAGTCCACATATAATTGACTCAGAAAAGAAAACTGATGAAGATGTAGCCTTTGAGGAGGAGGAGGAGGAGGAGGAGCTCGTTGCTTCAGCTACGAAGGCAGAGAACAGAATAAATGATATTTTGAATGAATTTCTCTCTGGTAATTGTGAAGATCTAGAAGGTGATCGAGCCATCAACATATTACAGGAGCGCTTGCAGATTAAACCTCTTACTTTAGAGAAATTATGCCTTCCAGATTTAGAAGCCATTCCAACAATGAATTTGAAATCTTCAAGAAGCAATCTATCAAAGCGTAGTTTGATCAGTGTGGACAATCAGTTACAAAAGATAGAAATTTTGAAATCTAAGCAGGACAATGTAAATTTGGTTAATCCTGTTTCTACACCATCATCAATGAGAAGTCCATTGGCATCGTTATCAGCACTAAATAGACGGATTTCACTTTCAAATTCATCAAGTGATTCATTTTCAGCTCATGGCATTGACCAATCTCCATCAAGAGATCCTTACCTTTTTGAACTCGGTAATCACTTATCTGATGCAGTTGGTAATACAGAGCAGTCAAGCGTTTCTAAGTTGAAGCCACTTTTAACCAGAGATGGTGGGACTGTAGCAAATGGAATTAAACCATCCAAAATTCTTTCTGGAGATGATTCCATGTCTAATATATCTTCAAGTAATATTTTAAATGTACCCCAAGTTGGGGGCAATACTGCTTTAAGTGGAACTTATGCCAGCACGGAGGCTAAAAATGTTAGTGTCAGCAGCACAGACGTGGAAATAAATGAGAAATTGAGTTGTCTTGAAGCCCAAGCAGATGCGGTGGCTAATATGCAGATTGAAGATCACGAAGGATCAGCTTCTGAGCAACCAAAATTATCTGAGGTGGATCTAATCAAAGAGTACCCGGTTGGCATTCGGAGTCAGTTGGATCAATCAGCTGCTACTTGTACTGAAAATATTGTTGATGGGTCATCTAGAAGCAGTGGTACAGAACACCGCGATGAGATGGAAGATCATGAAGGATCAGCTTCTGAGCAACCAAAGTCATCTAAGGTGGATGTGATTAAAGAGTACCCAGTAGCCATTCAGAGTCAGTTGGATCAATCAACTACTACTACTTGTGCTGAAAATATTGCCGATGGGGCATCTAGAAGCAGTGGAACGGATCACCATGATGGGGAACAGGTCAAGCCAAAATCTCGTGCAAACAAACAACACAAAGGCAAAAAGATTTCTCGGAGGCAAAGCCTTGCAGGTGCTGGTACAACGTGGCAAAGTGGGGTGAGAAGAAGTACCAGGTTCAAAACACGACCCTTGGAGTACTGGAAAGGTGAAAGGCTGTTGTACGGACGTGTACATGAGAGCCTGACGACAGTAATTGGGTTGAAGTATGTGTCTCCAGCAAAAGGAAATGGCAAACCAACCATGAAGGTGAAGTCTCTAGTCTCCAATGAGTACAAAGATCTCGTCGAGTTAGCAGCCCTTCACTGA

SEQ ID NO: 4 - protein, 2190 aa, Cucumis melo
ATGACAATGGTGAACGAAGAAACTCGACCCTCCGATGTAATCGATCCTCTTGCTGCTTATTCTGGTATCAATCTCTTTCCGACCGCATTTGGTACTTTGACGGATCCGTCAAAGCCACATGATCTTGGAACAGACCTCGACGGCATCCACAAGCGCCTCAAATCCATGGTGTTAAGGAGTCCCAGTAAACTATTAGAGCAGGCCAGATCAATATTAGATGGCAACTCAAAATCGATGATATCTGAAGCTGCTACATTTCTCGTGAAGAATGAGAAAAATGAGGCAGCTTCTGTGAAGGCAGAGGAAAATCCTCAAGAAAGAAGGCCGGCCTTAAACCGAAAGCGGGCTAGGTTTTCTTTAAAACCTGATGCTGGGCAACCTCCTGTGAACTTGGAACCAACATTTGACATCAAACAATTGAAAGACCCTGAGGAGTTCTTTTTGGCCTATGAAAAGCATGAAAATGCCAAAAAAGAAATCCAAAAACAGATGGGAGCAGTTTTAAAGGACTTGAACCAACAAAATCCATCGACAAATACACGCCAGCGTAGACCAGGGATTCTTGGGAGATCTGTTAGATACAAGCATCAATATTCATCAATAACAACTGAAGATGATCAGAATGTAGATCCTTCTCAAGTGACATTTGATTCAGGTGTTTTCAGTCCATTGAAATTGGGCACAGAAACACACCCAAGTCCACATATAATTGACTCAGAAAAGAAAACTGATGAAGATGTAGCCTTTGAGGAGGAGGAGGAGGAGGAGGAGCTCGTTGCTTCAGCTACGAAGGCAGAGAACAGAGTAAATGATATTTTGGATGAATTTCTCTCTGGCAATTGTGAAGATCTAGAAGGTGATCGAGCTATCAACATATTACAGGAGCGCTTGCAGATTAAACCCCTTACTTTAGAGAAATTATGCCTTCCAGATTTAGAAGCCATTCCAACAATGAATTTGAAATCTACAAGAGGCAATCTGTCAAAGCGTAGTTTGATCAGTGTGGACAATCAGTTACAAAAGACAGAAACCTTGAAATCTAAGGAGGACAATGAAAATTTGGTTAATCTTGTTTCTACACCATCATCAATGAGAAGTCCATTGGCATCATTATCAGCCCTAAATAGACGAATTTCACTTTCAAATTCATCAGGTGATTCATTTTCAGCTCATGGCATCGACCGATCTCCAGCAAGAGATCCTTACCTTTTTGAACTCGGTAATCACTTATCTGATGCAGTTGGTATTACAGAGCATTCAAGCGTTTCTAAGTTGAAGCCACTTTTAACCAGAGATGGTGGGACTATAGCAAATGGAATTCAACCATCCAAAATTCTTTCTGGAGACGATTCCATGTCTAAAATATCTTCAAGTAATATTTTAAATGTACTCCAAGTTGGTAGCAATACTGCTTTAAGTGGAACTTATGCCAGCACAGATGCTAAAAATGTTAGTGGGAGCAGCACAGACGTGGAAATAAATGAGAAATTAAGTTGTCTTGAAGCCCAAGCAGATGTGGTGGCTAATATGCAGATAGATCACCAAGGATCAGCTTCTGAGCAACCAAAATTATCTGAGGTGGATCTTATTGAAGAGTACCCGGTTGGCATTCGGAGTCAGTTGGATCAATCAGCTGCTACTTGTACTGAAAATATTGTTGATGGGTCGTCTAGAAGCAGTGGAACAGAACACCACGATGAGATGGAAGATCACGAAGGATCAGCTTCTGAGCAACCAAACTCATCTAAGGTGGATATGATTAAAGAGTACCCAGTCGGCATTCAGATTCAGTTGGATCAATCAACTACTACTACTACTTGTGCTGAAAAAATTGTCGATGGGACATCTAGAAGCAGTGGAACGGATCACCATGATGAGGAACAGGTCAAGCCAAAATCTCGTGCAAACAAACAACGTAAAGGCAAAAAGATTTCTGGGAGGCAAAGCCTTGCAGGTGCTGGTACAACGTGGAAAAGTGGGGTGAGAAGAAGTACCAGGTTCAAAATACGACCCTTGGAGTACTGGAAAGGTGAAAGGATGTTGTACGGACGTGTACATGAGAGCCTAGCGACAGTAATCGGGTTGAAGTATGTGTCTCCAGAAAAAGGAAATGGCAAACCAACCATGAAGGTGAAATCTCTAGTCTCCAATGAGTACAAAGATCTCGTCGACTTAGCAGCCCTTCACTGA

SEQ ID NO: 5 - protein, 154 aa, Cucumis sativus
MARARHPPRRKSNRTPSGSGAAQSSPTAPSTPLNGRTQNVRQAQNSSSRTIKKKKRFRPGTVALKEIRNLQKSWNLLIPASCFIRAVKEVSNQLAPQITRWQAEALVALQEAAEDFLVHLFEDTMLCAIHAKRVTIMKKDFELARRLGGKGRPW

SEQ ID NO: 6 - protein, 154 aa, Cucumis melo
MARARHPVQRKSNRTSSGSGAALSPPAVPSTPLNGRTQNVRKAQSPPSRTKKKKIRFRPGTVALREIRNLQKSWNLLIPASCFIRAVKEVSNQLAPQITRWQAEALVALQEAAEDFLVHLFEDTMLCAIHAKRVTIMKKDFELARRLGGKGRPW

SEQ ID NO: 7 - protein, 85 aa, Cucumis sativus
SRRQSLAGAGTTWQSGVRRSTRFKTRPLEYWKGERLLYGRVHESLTTVIGLKYVSPAKGNGKPTMKVKSLVSNEYKDLVELAALH

SEQ ID NO: 8 - protein, 85 aa, Cucumis melo
SGRQSLAGAGTTWKSGVRRSTRFKIRPLEYWKGERMLYGRVHESLATVIGLKYVSPEKGNGKPTMKVKSLVSNEYKDLVDLAALH